(19) United States
(12) Patent Application Publication
(10) Pub. No.: US 2005/0112151 A1
(43) Pub. Date: May 26, 2005

(54) SKIN ADHERENT HYDROGELS

(76) Inventor: Liou Liang Horng, Montville, NJ (US)

Correspondence Address: BLACKWELL SANDERS PEPER MARTIN LLP, 720 OLIVE STREET, SUITE 2400, ST. LOUIS, MO, 63101, US

(21) Appl. No.: 10/707,102

(22) Filed: Nov. 20, 2003

(51) Int. Cl. A61K 7/06; A61K 7/11; A61K 7/00

(52) U.S. Cl. 424/401; 424/70.15; 424/70.13

(57) ABSTRACT

There is disclosed hydrogel compositions comprising a combination of at least two of three components selected from non-acidic poly (N-vinyl lactam), chitosan derivatives and water-soluble multifunctional amine-containing polymers in various ratios. The hydrogels are useful as cavity-filling wound dressings, burn dressings, drug delivery systems, cosmetic masks, conductive electrodes, prostheses and wraps.

### BACKGROUND OF INVENTION

This invention relates to the field of gel formation using non-acidic poly(N-vinyl lactam), and chitosan derivatives or polyethyleneimine gels. Such gels can be employed as a pharmaceutical hydrogel for wound dressing, dental and local anesthetic. The gels are flexible and contour-conforming that can be used for a variety of other applications.

Poly(N-vinyl lactam) such as polyvinylpyrrolidone (PVP) has been used, for example, in pharmaceuticals, in certain types of films and in some cosmetic products and paper printing industries.

Chitosan is a deacetylated chitin, and is a linear polysaccharide of deacetylated N-acetyl-D-glucosamine. Chitosan has been used to absorb heavy metals from water and industrial waste streams, as a dyeing assistant in photographic emulsions, as cosmetic to absorb fat or grease from the oily faces, and as weight control ingredient to chelate fat or oil from the digesting system. Chitosan derivatives have also been used in cosmetics and conditioning agents in hair setting lotions and shampoos, when neutralized with acids as described in the U.S. Pat. Nos. 4,134,412 and 4,202,881. Polyethyleneimine can be used as fixing agents in the immobilization of enzyme preparations, lamination or sealing of food packaging items, sealing means for paper and paper board in contact with aqueous and fatty foods and in contact with dry food, prime coat for cellophane used for packaging food or impregnated in regenerated cellulose sheets, etc.

Polyvinylpyrrolidone has been known to form complexes with polyurethanes to yield hydrophilic blends or alloys. U.S. Pat. No. 4,646,730 describes a PVP/Silver Sulfadiazine hydrogel dressing in which electron beam radiation is required to cross-link the PVP and form a gel. In addition, magnesium trisilicate, hydrogen peroxide and/or polyacrylic acid are added for color stabilization.

As employed herein the term “acidic” with respect to poly(N-vinyl lactam) polymers means that the acidity is more than 1.4×10⁻² milliequivalents of carboxylic acid groups per gram of polymer and the term “non-acidic” with respect to poly(N-vinyl lactam) polymers means acidity is less than 1.4×10⁻² milliequivalents (hereinafter “m.e.”) of carboxylic acid groups per gram of polymer.

U.S. Pat. Nos. 5,420,197 and 5,258,421 describe acidic (acidity is more than 1.4×10⁻² milliequivalents (hereinafter m.e.) of carboxylic acid groups per gram of polymer) poly(N-vinyl lactam)-chitosan derivative gels wherein the K values of PVP are at least 60. U.S. Pat. No. 6,379,702 describes acidic poly(N-vinyl lactam)-chitosan derivative gels wherein the acidic PVP has a K value of less than 60. U.S. Pat. No. 5,306,504 describes acidic poly(N-vinyl lactam)-polyethyleneimine gels wherein the K values of the PVP are at least 50. In these patents, the concept of acidic (as defined above) ring-opened pyrrolidone groups was made use of to attain the absorbent gels. Thermal treatment of the non-acidic (acidity is less than 1.4 times 10⁻² m.e. of carboxylic acid groups per gram of polymer) PVP to make acidic ring-opened pyrrolidone groups was described in these patents. It is apparent that there would be an advantage in making skin-adhering gels in the absence of expensive equipment, thermal treatment for ring opening, and/or processing. Other patents disclosing poly(N-vinyl lactam) based gels are U.S. Pat. No. 5,645,855 (ring opened) and U.S. Pat. No. 6,365,664 (aldehyde grafted PVP).

The dry socket is a most painful experience for the unfortunate patient who has the condition. A dry socket is a condition where the healing blood clot has been prematurely dislodged from a dental extraction site. The socket or alveolus remaining has only sensitive bony walls that are exposed to uncomfortable temperatures and stimulants such as beverages or cold air. The current treatment in the U.S. for the malady is to rinse the socket with saline and then place a sedative dressing into the socket. The sedative dressing may either be sterile gauze type material or sterile gel foam coated with Eugenol or some other abundant medicament. The purpose for this treatment modality is two fold. The first purpose is to alleviate the discomfort associated with the exposed nerves found in the bony walls of the socket. The medicament usually contains some chemical agent that will desensitize the area and give the patient relief. The second purpose of the medicated gauze is to protect the natural healing of the socket from the constant rinsing of the area from the patient's own saliva. This protection allows cells to migrate undisturbed into the socket and commence secondary healing. Most textbooks recommend the removal and replacement of the gauze after three days. Therefore the current treatment may require not one but two painful visits to the dentist following the original treatment.

Dry socket is a quite common occurrence in the U.S. Approximately 30% of all 3^{rd} molar extractions return with diagnosis of dry socket. This number is a result of the extraction occurring in the highly trained oral surgeon's offices. The incidence for dry socket occurrence following the extraction of non-third molar teeth is not as high as 30% but is still high enough to be a common occurrence in most dental practices. The problem is exacerbated by non-compliance on the part of the patient. Many patients rinse too aggressively, smoke cigarettes, or use alcoholic beverages following dental extractions. These behaviors contribute to many of the dry socket cases. Patients in the U.S. have come to depend on affordable, pain-free dental and medical services.

A more efficient, less painful treatment of dental dry socket with fewer steps or visits to the dentist is therefore desired. The creation of a temporary “artificial clot” in the area of the dry socket such that the wound would heal on its own would be a welcome advance in the treatment of the dental dry socket. The artificial clot will provide temporary protection of the socket and remain long enough to provide the commencement of natural healing to the point that the area cell growth would be self-sufficient. Another advantage of such an artificial clot would be to also provide a sedative effect on the exposed bone. Therefore, the product should have a fairly strong analgesic effect that would be sufficiently long acting to provide relief from the acute pain and still safe enough to remain intraorally for the 24-48 hour time frame.

It is the principal object of the present invention to provide an improved skin adhesive hydrogel composition based on non-acidic (acidity is less than 1.4 times 10⁻² m.e. of carboxylic acid groups per gram of polymer) polyvinylpyrrolidone not requiring the thermal treatment for ring opening of the pyrrolidone groups.

It is another object of the present invention to provide a skin adhesive hydrogel based on hydrogen binding of the imine (such as polyethyleneimine) and carboxyl (such as carboxymethyl chitosan) groups not requiring the use of ionizing radiation or thermal treatment for ring opening in its preparation.

It is still another object of the present invention to provide dental and local anesthetic hydrogel formulations that have certain advantages over other topical anesthetics.

It is another further object to provide gels using non-acidic poly(N-vinyl lactam) and chitosan derivatives or amine-containing polymer to be used in a variety of products such as cavity dressings, drug delivery patches, face masks, wound dressings and prostheses, etc.

### SUMMARY OF INVENTION

The present invention is a skin adhesive hydrogel composition and treatment methods employing such compositions that perform these important objects to help promote the healing of such injuries as the dental dry socket condition as well as providing treatment of numerous other body wounds. The present invention provides dental and medical providers with compositions that supply patients with a less painful alternative to the standard gauze packing treatment.

Skin adherent compositions of this invention comprise a gel formed by a mixture of two or more of (1) a non-acidic poly N-vinyl lactam with a K value of 30 or higher (2) a water soluble multifunctional amine-containing polymer and mixtures thereof or (3) a chitosan derivative or mixtures thereof.

A first typical skin adhesive therapeutic hydrogel composition of the present invention comprises a mixture of:
1. a water-soluble multifunctional amine-containing polymer;
2. non-acidic poly(N-vinyl lactam) with K values of 30 or above;
3. with or without plasticizer(s), moisturizer(s), drug(s) or other bio-effecting or body-treating material(s).

The second typical gel system composition comprises a mixture of:
1. a water-soluble multifunctional amine-containing polymer;
2. a chitosan derivative;
3. with or without plasticizer(s), moisturizer(s), drug(s) or other bio-effecting or body-treating material(s), forming a skin adhesive or cosmetic hydrogel.

The third typical gel system composition comprises a mixture of:
1. non-acidic poly(N-vinyl lactam) with K values of 30 or above;
2. a chitosan derivative;
3. with or without plasticizer(s), moisturizers(s), drugs(s) or other bio-effecting or body-treating materials.

A skin adhesive, therapeutic or cosmetic hydrogel was formed.

Preferred, but non-limiting, applications of the compositions of this invention are cavity-filling wound dressings, burn dressings, drug delivery systems, cosmetic masks, conductive electrode, prostheses and wraps, and the like.

### DETAILED DESCRIPTION

It has been found that the non-acidic poly(N-vinyl lactam) such as polyvinylpyrrolidone (PVP) exhibits a pH of about 7 where acidity is less than 1.4×10⁻² m.e. of carboxylic acid groups per gram of polymer. Such polymers form hydrophilic gels with either chitosan derivatives or amine-containing polymers. The amine-containing polymer can also form gels with chitosan derivatives. The gels are flexible and transparent or translucent and may be used alone or with various additives. The gels can be used for cavity packing, wound and burn dressings, drug delivery systems, prostheses, cosmetic masks and nail wraps, and other applications where the high absorption capacity of the gel and the high heat capacity and transport capacity of water as part of the hydrophilic gel can be utilized. These gels may have either a tacky quality or non-tacky quality. Anesthetics can be integrated into the hydrogel system to make the dental and/or local topical anesthetic gel.

The commercially available non-acidic (pH is about 7 where acidity is less than 1.4×10⁻² m.e. of carboxylic acid groups per gram of polymer) poly(N-vinyl lactam) has a K value of 30 120. The K value represents kinetic viscosity. It is a measure of the resistive flow of a fluid under the influence of gravity. The K value is derived from viscosity measurements and is calculated according to Eikentscher's formula described by Kline, G. M., “Polyvinylpyrrolidone”, Modern Plastics p 157 (November 1945) and is also described in General Aniline & Film Corporation Technical Bulletin 7583-033. The K value is a function of molecular weight.

The term poly(N-vinyl lactam) as used herein means homopolymers, copolymers and terpolymers of N-vinyl lactams such as N-vinylpyrrolidone, N-vinylbutyrolactam, N-vinylcaprolactam, and the like, as well as the foregoing prepared with minor amounts, for example, up to about 50 weight percent, of one of a mixture of other vinyl monomers copolymerizable with the N-vinyl lactams. Copolymers or terpolymers of poly (N-vinyl-lactam) may comprise N-vinyl-lactam monomers such as vinylpyrrolidone copolymerized with monomers containing a vinyl functional group such as acrylates, hydroxyalkylacrylates, methacrylates, acrylic acid or methacrylic acid, and acrylamides. Of the poly(N-vinyl lactam) homopolymers, the polyvinylpyrrolidone (PVP) homopolymers are preferred. Of the poly(N-vinyl lactam) copolymers, the vinyl pyrrolidone and acrylamide copolymers are preferred. Of the poly(N-vinyl lactam) terpolymers, the vinylpyrrolidone, vinylcaprolactam, dimethylaminoethyl methacrylate terpolymers are preferred. A variety of polyvinylpyrrolidones are commercially available.

It is a noted advantage of this invention that ring opening of the non-acid poly(N-vinyl lactam) polymers of this invention is omitted. Contrary to the prior art, stable, useful hydrogels are provided in accordance with this invention with non-acidic poly(N-vinyl lactam) polymers thus providing further economy of production of hydrogels.

Chitosan, a natural product, is derived from chitin. Chitin is an unbranched linear polysaccharide of N-acetyl-D-glucosamine units linked by beta-1,4 bonds. It is a polymer of glucose in which the hydroxyl group on C-2 is replaced by the N-acetylamino group-NHCOCH₃. In chitosan, the acetyl group is absent. Therefore, chitosan is a deacetylated chitin. Chitosan contains approximately 7% nitrogen and is structurally similar to cellulose. Chitin occurs in nature in the exoskeletons of arthropods such as crabs, lobsters and shrimp. Chitin can be obtained from these sources as an amorphous powder or flakes after dissolution of the calcium carbonate with mineral acids and removal of the proteins. It is also found in some fungi, algae and yeast.

Chitosan becomes soluble in water when protonated with acids. The polymer thus formed is positively charged and thus more likely to interact with negatively charged surfaces like skin and hair.

Chitosan derivatives are commercially available as, for example, chitosan neutralized with pyrrolidone carboxylic acid, carboxymethyl sodium salt of chitosan, chitosan neutralized with glutamic acid, N,O-carboxymethyl chitosan, etc. Suitable chitosan derivatives for this invention are the biocompatible salts of chitosan such as those with pyrrolidone carboxylic acid, glutamic acid, acetate, N,O-carboxymethyl chitosan, and N,O-carboxybutyl chitosan, and the like.

The multifunctional amine-containing polymer is a water-soluble polymer containing basic amine groups. Examples are polyethyleneimine, amine terminated polyethylene oxide polymers, amine terminated polyethylene/polypropylene oxide polymers, polymers and copolymers of dimethyl amino ethyl methacrylate, and vinyl pyrrolidones and the like.

The gel may be prepared by dissolving the non-acidic poly(N-vinyl lactam) such as polyvinylpyrrolidone in aqueous solution, then adding an aqueous solution of chitosan derivatives or amine-containing polymer with sufficient agitation to attain a homogenous mixture. The solvent used for the gel preparation is preferably substantially aqueous. For example, the gels may be prepared in water or in hydroalcohols such as water/isopropyl alcohol, or water/ethanol, or water/polyethylene glycol, etc. The gel can also be prepared by dissolving the chitosan derivatives in aqueous solution, then adding an aqueous solution of amine-containing polymer with sufficient agitation to attain a homogenous mixture. Gel formation, in this instance, may be less quickly formed than with other embodiments of this invention.

The proportions of the non-acidic (acidity is less than 1.4×10⁻² m.e. of carboxylic acid groups per gram of polymer) PVP to multifunctional amine-containing or chitosan derivatives polymer may vary widely. Generally, the proportion, by weight, for the PVP/polyethyleneimine gel is between 2/1 to about 80/1. Also, the proportion for the PVP/chitosan derivative gel is generally between 2/1 to about 100/1. The proportion, by weight, of the chitosan derivatives to multifunctional amine-containing polymer gel is generally between 50/1 and 1/50. The total polymer concentration as well as the ratios of the two polymer components at which the gel is made shows an effect on the consistency of the gel, which becomes softer at lower concentrations. The gel may be made with a total polymer content ranging from about 5 to about 95 wt. % solids, preferably from about 8 wt. % to about 35 wt. % solids. The blend may be allowed to cure for a time from a few seconds to a few hours. The time and temperature for curing are not critical. For purposes of convenience, ambient temperature may be used but the time can be shortened at elevated temperatures. Elevated temperatures in the range of from about 40° C. to about 100° C. have been found to be useful not only for drying but also to shorten the time for curing the gel.

The gels are stable and maintain their physical integrity after absorbing large quantities of liquid. The gels can be sterilized by radiation sterilization, autoclave or exposed to ethylene oxide. The gels are hydrophilic and capable of absorbing many times of their dry weight in water. While the exact nature of the mechanism by which the gel forms is not known, and while it is not intended to be bound by theory, it is believed to be caused by hydrogen and ionic bonds between the amine and carboxyl groups of the gel mixture.

Wetting, dispersing agents or surfactants as are known in the art may be added. Glycerin in an amount of 0 to 50 wt. %, preferably from about 5 to 40 wt. % may be added to the gel to increase tack, pliability after drying for the gel. Propylene glycol or polyethylene glycol may also be added.

Other additives may be combined with the hydrogels of this invention including organic salts, inorganic salts, alcohols, amines, polymer lattices, fillers, surfactants, pigments, dyes, fragrances, etc. as long as they don't interfere with gel formation. Many of these materials can be released from the gel.

The gels of this invention are especially useful as carriers for a wide range of pharmaceutically acceptable and releasable biologically active agents having curative or therapeutic value for human or non-human animals. Included among the biologically active materials which are suitable for incorporation into the gels of the invention are hypnotics, sedatives, tranquilizers, anti-convulsants, muscle relaxants, analgesics, antipyretic agents, anti-inflammatory agents, local anesthetics, antispasmodics, anti-ulcer agents, anti-virals, anti-bacterials, anti-fungals, sympathomimetic agents, cardiovascular agents, antitumor agents, etc. Particularly biologically active additives are nitroglycerine, scopolamine, pilocarpine, ergotamine tartrate, phenylpropanolamine, theophylline, antimicrobials tetracycline, neomycin, oxytetracycline, triclosan, sodium cefazolin, silver sulfadiazine, salicylates such as methylsalicylate and salicylic acid, nicotinates such as methyl nicotinate, menthol, capsicum and benzocaine. Hydrating agents such as sodium pyrrolidone carboxylic acid may be added. The large amount of water in the gel can also serve a hydrating function to the skin.

The gels of this invention can be employed to make adsorbent wound or burn dressings or packing or filling, skin masks or wraps, drug delivery patches, prosthetic devices, implants and dry film products, etc. In addition to the incorporation of a plasticizer and surfactant in the gel, the gel may contain a bactericide such as chlorhexidine gluconate, silver or copper compounds, or an antibiotic or antimicrobial. The gel may also contain sodium chloride, or potassium chloride, or sodium bicarbonate, or other salts to match physiological saline in order to prevent osmotic pumping from the wound, as well as agents to promote regrowth of tissue.

Electrolyte salts may be included in the gel to make the gel conductive for use in attaching electrocardiogram electrodes, transcutaneous electrical nerve stimulator electrodes, electro-surgical unit electrodes, biofeedback electrodes and iontophoresis drug delivery electrodes and defibrillation pads. Sodium chloride, potassium chloride and magnesium acetate are examples of suitable electrolyte salts.

The hydrogel of this invention, because of its high water content may be used to hydrate the skin and provide a cooling effect for cosmetic applications. The addition of skin moisturizers like sodium pyrrolidone carboxylate, lactic acid, hyaluronic acid and hydrolyzed collagen, preservatives such as butylated toluenes, colorant and odorants and other agents can provide further action on the skin.

The gels are biocompatible, able to conform to a wound cavity, non-adhere to the wound, can absorb exudates, removable in one piece from the wound and hold its physical integrity when swollen with exudates and provide comparative ease in handling. The gel has excellent hydrating capacity and can be easily removed and cleaned when used as facemask. The gel can be made into a flexible, clear, hydrophilic film that adheres to the skin when wetted with water. The film can be easily peeled off after a period of time without leaving residues.

The gels can be packaged by itself in a mold, in a dry film form to be wetted before use, or as a two-component system which requires mixing in accordance with this invention prior to use, or may be provided on a substrate and covered with a release liner as a roll stock and the release liner can be removed prior to application to the skin. The gel may be coated or spread onto unlimited variety of backing or substrate by any means known in the art. The substrate selection to the desired properties such as reinforcement, gas and liquid barriers, air permeability, protection or selection of the area of treatment, etc., are known to those skilled in the art.

The gels may be used in cosmetic preparations such as facemasks and nail/joint wraps. The gel serves a hydrating function with or without a backing and a cosmetic effect may be enhanced with the incorporation of other ingredients. A kit for a cosmetic gel may comprise a ready-made gel or two components such as a poly(N-vinyl lactam) component and a chitosan derivative or amine containing polymer component. Other cosmetic agents such as hydrating agents, fragrances, and skin nutrients, etc. are also supplied to the ready-made gel or to either component or as the third component. For use, the components may be mixed and applied. The gel can be easily peeled off after use. The cosmetic applications are intended to enhance or improve physical appearance.

In the dental and/or local anesthetic application, the hydrogel comprises lidocaine, or benzocaine, or Eugenol, etc., about 1 to 30 wt. % preferably 2 to 20 wt. %, and aqueous non-acidic poly(N-vinyl lactam) solution and amine-containing polymer (such as polyethyleneimine) in a ratio of from about 80/1 to about 2/1, preferably from about 30/1 to about 5/1. To this mixture is added moisturizer(s) from about 0 to 50 wt. % preferably 5 to 25 wt. %, and preservative(s) from about 0 to 4 wt. % preferably 0.01 to 2 wt. %, to form a blend at about 3 wt. % to 90 wt. % total solid concentration, preferably from about 10 wt. % to about 70 wt. % total solid concentration. The mixture is allowed to cure until a gel is formed.

An alternate dental and/or local anesthetic application comprises:
(a) from about 1 to about 30 wt. %, preferably from about 2 to about 20 wt. %, of a suitable local anesthetic such as lidocaine, benzocaine, or Eugenol, and the like;
(b) an aqueous mixture of a non-acidic poly(N-vinyl lactam) solution and an amine-containing polymer (such as chitosan derivatives) from about 80/1 to about 2/1, preferably from about 30/1 to about 5/1;
(c) moisturizer(s) and plasticizer(s) of from about 0 to about 50 wt. % preferably 5 to 25 wt. %, and
(d) preservative(s) from about 0 to about 4 wt. % preferably 0.01 to 2 wt. %, to form a blend at about 3 wt. % to 90 wt. % total solid concentration, preferably from about 10 wt. % to about 70 wt. % total solid concentrations. The mixture is allowed to cure until a gel is formed.

The following examples are intended to illustrate but not limit the invention.

### EXAMPLE 1

Fifty grams of a 35% by weight aqueous solution of non-acidic K60 PVP at pH 7 was mixed with twenty grams of 10% by weight aqueous solution of polyethyleneimine. A gel was immediately formed.

### EXAMPLE 2

Fifty grams of a 35% by weight aqueous solution of non-acidic K60 PVP at pH 7 was mixed with fifty grams of 2% by weight carboxymethyl chitosan aqueous solution. A gel is formed after 60 seconds.

### EXAMPLE 3

Fifty grams of a 2.4% by weight aqueous solution of polyethyleneimine was mixed with fifty grams of 2% by weight aqueous solution of carboxymethyl chitosan solution. A gel was formed in less than 20 minutes.

### EXAMPLE 4

Fifty grams of a 0.6% by weight aqueous solution of polyethyleneimine was mixed with fifty grams aqueous solution of 2% by weight carboxymethyl chitosan and 15 grams of glycerin. A gel was formed in less than 5 minutes.

### EXAMPLE 5

Fifty grams of a 4% by weight aqueous solution of carboxymethyl chitosan was mixed with fifty grams of 94% by weight glycerin and 0.6 grams of 50% by weight polyethyleneimine aqueous solution. A gel was formed in less than 5 minutes.

### EXAMPLE 6

Seventy-nine grams of a 40% by weight of non-acidic K60 PVP at pH 7 and 21 grams of glycerin in aqueous solution was mixed with 2 grams of carboxymethyl chitosan, 47 grams of glycerin (aqueous solution), 0.6 grams of 50% by weight of polyethyleneimine aqueous solution and 51 grams of water. A gel was formed in less than 5 minutes.

### EXAMPLE 7

A mixture was formed containing:
(a) 91 grams of a 37.5% by weight of non-acidic K60 PVP at pH 7
(b) 8 grams of lidocaine hydrogen chloride,
(c) 1.0 grams of 50% by weight of glutaric dialdehyde aqueous solution.

The above mixture was then mixed with:
(a) 90.5 grams of 8.2% by weight of polyethyleneimine,
(b) 5 grams of glycerin,
(c) 4 grams of polyvinylpyrrolidone/dimethylaminoethyl-methacrylate copolymer and
(d) 0.5 gram spearmint oil.

A gel is formed in less than one minute. The gel can be applied as the dental anesthetic and/or as a local anesthetic hydrogel.

### EXAMPLE 8

A mixture was formed containing:
(a) 93 grams of a 37.5% by weight aqueous solution of non-acidic K60 PVP at pH 7,
(b) 6 grams of lidocaine hydrogen chloride and
(c) 1.0 grams of 50 wt. % glutaric dialdehyde aqueous solution.

The above mixture was then mixed with:
(a)100 grams of 7.5% by weight of polyethyleneimine,
(b)₅ grams of glycerin,
(c)₄ grams of poly vinylpyrrolidone/dimethylaminoethyl-methacrylate copolymer and (d)0.5 grams of spearmint oil.

A gel is formed in less than one minute. The gel can be applied as the dental anesthetic and/or local anesthetic hydrogel.

### EXAMPLE 9

A mixture was formed containing:
(a)₇₅ grams of a 37.5% by weight of non-acidic K60 PVP at pH 7,
(b) 7 grams of benzocaine,
(c) 1.0 gram of a 50% aqueous solution of glutaric dialdehyde and
(d)17 grams of polyethylene glycol aqueous solution.

The above mixture was then mixed with: (a) 50 grams of 8% by weight of polyethyleneimine, (b) 10 grams of benzocaine, (c) 10 grams of glycerin, (d) 8 grams of poly vinylpyrrolidone/dimethylaminoethyl-methacrylate copolymer, (e) 0.5 grams of spearmint oil and (f) 22 grams of polyethylene glycol.

A gel is formed in less than one minute. The gel can be applied as the dental anesthetic and/or local anesthetic hydrogel.

There has been described a novel hydrogel of general application. While the hydrogels of this invention have been described with reference to specific compounds and examples no intention is made by such reference to limit the scope of this invention unless expressly stated. Various modifications may be made in the materials and sequence of process steps as well as process combinations, which are adapted to suit the various reactants and products without departing from this invention. The foregoing description is given for clarity of understanding only and no unnecessary limitations should be understood there from, as modifications will be obvious to those skilled in the art.

1. A composition comprising a hydrogel formed by a mixture of two or more of (1) a non-acidic poly(n-vinyl lactam) with a k value of at least 30 (2) a water soluble multifunctional amine-containing polymer and mixtures thereof or (3) a chitosan derivative or mixtures thereof.

2. The composition of claim 1 wherein the non-acidic poly(N-vinyl lactam) is selected from the group consisting of homopolymers, coplymers and terpolymers of N-vinyl lactam.

3. The composition of claim 2 wherein the copolymers and terpolymers of poly(N-vinyl lactam) are selected from the group consisting of N-vinyl lactam monomer copolymerized with monomers containing a vinyl functional group.

4. The composition of claim 3 wherein the vinyl containing monomers are selected from the group consisting of acrylates, hydroxyalkylacrylates, methacrylates, acrylic acid, methacrylic acid and acrylamindes.

5. The compositions of claim 4 wherein the vinyl containing monomers are selected from the group consisting of vinylpyrrolidone, vinylcaprolactam, dimethylaminoethyl methacrylate terpolymers.

6. The composition of claim 2 wherein a homopolymer of the non-acidic poly(N-vinyl lactam) is vinylpyrrolidone.

7. The composition of claim 1 wherein the multifunctional amine-containing polymer is selected from the group consisting of polyethyleneimine, amine terminated polyethylene oxide polymers, amine terminated polyethylene/polypropylene oxide polymers, polymers and copolymers of dimethyl amino ethyl methacrylate, and vinyl pyrrolidone.

8. The composition of claim 1 wherein the chitosan derivative is a biocompatible salt.

9. The composition of claim 8 wherein the chitosan salt is selected from the group consisting of chitosan reacted with a reactant selected from the group consisting of pyrrolidone carboxylic acid, glutamic acid and an acetate.

10. The composition of claim 1 wherein the chitosan is selected from the group consisting of N,O-carboxymethyl chitosan, and N,O-carboxybutyl chitosan.

11. The composition of claim 1 further including biologically active and pharmaceutically acceptable substances having curative or therapeutic value.

12. The composition of claim 11 wherein the active materials are selected from the group consisting of hypnotics, sedatives, tranquilizers, anti-convulsants, muscle relaxants, analgesics, antipyretic agents, anti-inflammatory agents, local anesthetics, antispasmodics, anti-ulcer agents, anti-virals, anti-bacterials, anti-fungals, sympathomimetic agents, cardiovascular agents and antitumor agents.

13. The composition of claim 12 wherein said active materials are selected from the group consisting of nitroglycerine, scopolamine, pilocarpine, ergotamine tartrate, phenylpropanolamine, theophylline, antimicrobials tetracycline, neomycin, oxytetracycline, triclosan, sodium cefazolin, silver sulfadiazine, methylsalicylate, salicylic acid, nicotinates, methyl nicotinate, chlorhexidine gluconate, menthol, capsicum, lidocaine and benzocaine.

14. The composition of claim 1 further including an electrolyte whereby said gel is rendered electrically conductive.

15. The composition of claim 14 wherein said electrolyte is selected from the group consisting of sodium chloride, potassium chloride and magnesium acetate.

16. The composition of claim 1 further including a skin-hydrating agent.

17. The composition of claim 16 wherein said skin-hydrating agent is selected from the group consisting of water, sodium pyrrolidone carboxylate, lactic acid, hyaluronic acid and hydrolyzed collagen.

18. The composition of claim 1 further including enhancing agents selected from the group consisting of wetting agents, moisturizers, plasticizers, surfactants and dispersing agents.

19. The composition of claim 18 where said agents are selected from the group consisting of glycerin, propylene glycol and polyethylene glycol.

20. The composition of claim 1 further including an electrolytic salt as an anti-osmotic agent.

21. The composition of claim 20 wherein said agent is selected from the group consisting of an alkali metal chloride and sodium bicarbonate.

22. The composition of claim 1 further including additives selected from the group consisting of polymer lattices, fillers, surfactants, pigments, dyes and fragrances.

23. (EXAMPLE 6) The composition of claim 1 formed by combining (a) an aqueous solution comprising non-acidic polyvinylpyrrolidone and glycerin in a weight ratio of about 1.5 to 1 with (b) an aqueous solution comprising carboxymethyl chitosan, glycerin and polyethyleneimine in a weight ratio, respectively, of about 6.6 to 156 to 1 wherein the total weight of aqueous solutions (a) and (b) are in the range of about 1 to 1.

24. A composition comprising a hydrogel formed by a mixture of a non-acidic poly(N-vinyl lactam) with a K value of at least 30 at and a water soluble multifunctional amine-containing polymer.

25. The composition of claim 24 wherein the poly(N-vinyl lactam is selected from the group consisting of homopolymers, coplymers and terpolymers of N-vinyl lactam.

26. The composition of claim 25 the copolymers and terpolymers of poly(N-vinyl lactam) are selected from the group consisting of N-vinyl lactam monomer copolymerized with monomers containing a vinyl functional group.

27. The composition of claim 26 wherein the vinyl containing monomers are selected from the group consisting of acrylates, hydroxyalkylacrylates, methacrylates, acrylic acid, methacrylic acid and acrylamindes.

28. The composition of claim 24 wherein a homopolymer of the non-acidic poly(N-vinyl lactam) is vinylpyrrolidone.

29. The composition of claim 25 wherein the weight ratio of poyvinylpyrrolidone to multifunctional amine-containing polymer is in the range of from about 2/1 to about 80/1.

30. The composition of claim 24 wherein the multifunctional amine-containing polymer is selected from the group consisting of polyethyleneimine, amine terminated polyethylene oxide polymers, amine terminated polyethylene/polypropylene oxide polymers, polymers and copolymers of dimethyl amino ethyl methacrylate, and vinyl pyrrolidone.

31. The composition of claim 24 further including biologically active and pharmaceutically acceptable substances having curative or therapeutic value.

32. The composition of claim 24 wherein the active materials are selected from the group consisting of hypnotics, sedatives, tranquilizers, anti-convulsants, muscle relaxants, analgesics, antipyretic agents, anti-inflammatory agents, local anesthetics, antispasmodics, anti-ulcer agents, anti-virals, anti-bacterials, anti-fungals, sympathomimetic agents, cardiovascular agents and antitumor agents.

33. The composition of claim 32 wherein said active materials are selected from the group consisting of nitroglycerine, scopolamine, pilocarpine, ergotamine tartrate, phenylpropanolamine, theophylline, antimicrobials tetracycline, neomycin, oxytetracycline, triclosan, sodium cefazolin, silver sulfadiazine, methylsalicylate, salicylic acid, nicotinates, methyl nicotinate, chlorhexidine gluconate, menthol, capsicum, lidocaine and benzocaine.

34. The composition of claim 24 further including an electrolyte whereby said gel is rendered electrically conductive.

35. The composition of claim 34 wherein said electrolyte is selected from the group consisting of sodium chloride, potassium chloride and magnesium acetate.

36. The composition of claim 24 further including a skin-hydrating agent.

37. The composition of claim 36 wherein said skin-hydrating agent is selected from the group consisting of water, sodium pyrrolidone carboxylate, lactic acid, hyaluronic acid and hydrolyzed collagen.

38. The composition of claim 24 further including enhancing agents selected from the group consisting of wetting agents, moisturizers, plasticizers, surfactants and dispersing agents.

39. The composition of claim 38 where said agents are selected from the group consisting of glycerin, propylene glycol and polyethylene glycol.

40. The composition of claim 24 further including an electrolytic salt as an anti-osmotic agent.

41. The composition of claim 40 wherein said agent is selected from the group consisting of an alkali metal chloride and sodium bicarbonate.

42. The composition of claim 24 further including additives selected from the group consisting of polymer lattices, fillers, surfactants, pigments, dyes and fragrances.

43. A composition comprising a non-acidic poly(N-vinyl lactam) with a K value of at least 30 and a multifunctional amine-containing polymer in a weight ratio of about 8.75 to 1.

44. (EXAMPLE 8) A composition formed by combining (a) an aqueous solution comprising non-acidic polyvinylpyrrolidone, lidocaine hydrogen chloride and glutaric dialdehyde in a weight ratio, respectively, of about 69.7 to 12 to 1 with (b) an aqueous solution comprising polyethyleneimine, glycerin and polyvinylpyrrolidone/dimethylaminoethyl-methacrylate copolymer in a weight ratio, respectively, of about 1.87 to 1.25 to 1 wherein the total weight of aqueous solution of each of (a) and (b) are in the range of about 0.9 to 1 respectively.

45. (EXAMPLE 7) A composition formed by combining (a) an aqueous solution comprising non-acidic polyvinylpyrrolidone, lidocaine hydrogen chloride and glutaric dialdehyde in a weight ratio, respectively, of about 68.2 to 16 to 1 with (b) an aqueous solution of comprising polyethyleneimine, glycerin and polyvinyl pyrrolidone/dimethylaminoethyl-methacrylate copolymer in a weight ratio, respectively, of about 1.85 to 1.25 to 1 wherein the total weight of the aqueous solution in each of (a) and (b) are in the range of about 1 to 1 respectively.

46. (EAMPLE 9) A composition formed by combining (a) an aqueous solution comprising non-acidic polyvinylpyrrolidone, polyethylene glycol, benzocaine and glutaric dialdehyde in a weight ratio respectively of about 56 to 34 to 14 to 1 with (b) an aqueous solution comprising polyethylene glycol, glycerin, benzocaine, polyvinyl pyrrolidone/dimethylaminoethyl-methacrylate copolymer and polyethyleneimine in the weight ratio respectively of about 5.5 to 2.5 to 2.5 to 2 to 1 wherein the total weight of each aqueous solution (a) and (b) is in the range of about 1 to 1.

47. A composition comprising a hydrogel formed by a mixture of a non-acidic poly(N-vinyl lactam) with a K value of at least 30 and a chitosan derivative or mixtures thereof.

48. The composition of claim 47 wherein the non-acidic poly(N-vinyl lactam) is selected from the group consisting of homopolymers, coplymers and terpolymers of N-vinyl lactam.

49. The composition of claim 48 wherein the copolymers and terpolymers of poly(N-vinyl lactam) are selected from the group consisting of N-vinyl lactam monomer copolymerized with monomers containing a vinyl functional group.

50. The composition of claim 49 wherein the copolymers and terpolymers of poly(N-vinyl lactam) are selected from the group consisting of N-vinyl lactam monomer copolymerized with monomers containing a vinyl functional group.

51. The composition of claim 50 wherein the vinyl containing monomers are selected from the group consisting of vinylpyrrolidone, vinylcaprolactam, dimethylaminoethyl methacrylate terpolymers.

52. The composition of claim 48 wherein a homopolymer of the non-acidic poly(N-vinyl lactam) is vinylpyrrolidone.

53. The composition of claim 47 wherein the chitosan derivative is a biocompatible salt.

54. The composition of claim 53 wherein the chitosan salt is selected from the group consisting of chitosan reacted with a reactant selected from the group consisting of pyrrolidone carboxylic acid, glutamic acid and an acetate.

55. The composition of claim 1 wherein the chitosan is selected from the group consisting of N,O-carboxymethyl chitosan, and N,O-carboxybutyl chitosan.

56. The composition of claim 55 further including biologically active and pharmaceutically acceptable substances having curative or therapeutic value.

57. The composition of claim 56 wherein the active materials are selected from the group consisting of hypnotics, sedatives, tranquilizers, anti-convulsants, muscle relaxants, analgesics, antipyretic agents, anti-inflammatory agents, local anesthetics, antispasmodics, anti-ulcer agents, anti-virals, anti-bacterials, anti-fungals, sympathomimetic agents, cardiovascular agents and antitumor agents.

58. The composition of claim 57 wherein said active materials are selected from the group consisting of nitroglycerine, scopolamine, pilocarpine, ergotamine tartrate, phenylpropanolamine, theophylline, antimicrobials tetracycline, neomycin, oxytetracycline, triclosan, sodium cefazolin, silver sulfadiazine, methylsalicylate, salicylic acid, nicotinates, methyl nicotinate, chlorhexidine gluconate, menthol, capsicum and benzocaine.

59. The composition of claim 47 further including an electrolyte whereby said gel is rendered electrically conductive.

60. The composition of claim 59 wherein said electrolyte is selected from the group consisting of sodium chloride, potassium chloride and magnesium acetate.

61. The composition of claim 47 further including a skin-hydrating agent.

62. The composition of claim 61 wherein said skin-hydrating agent is selected from the group consisting of water, sodium pyrrolidone carboxylate, lactic acid, hyaluronic acid and hydrolyzed collagen.

63. The composition of claim 47 further including enhancing agents selected from the group consisting of wetting agents, moisturizers, plactisizers, surfactants and dispersing agents.

64. The composition of claim 63 where said agents are selected from the group consisting of glycerin, propylene glycol and polyethylene glycol.

65. The composition of claim 47 further including an electrolytic salt as an anti-osmotic agent.

66. The composition of claim 65 wherein said agent is selected from the group consisting of an alkali metal chloride and sodium bicarbonate.

67. The composition of claim 47 further including additives selected from the group consisting of polymer lattices, fillers, surfactants, pigments, dyes and fragrances.

68. The composition of claim 47 wherein the weight ratio of the poly(N-vinyl lactam) to chitosan derivative is in the range of from about 2/1 to about 100/1.

69. The composition of claim 47 wherein comprising, by weight, from about 1 percent to about 30 percent of a pharmaceutically acceptable local anesthetic, from about 0 percent to about 50 percent of a moisturizer, from about 0 percent to about 50 percent of a plasticizer, from about 0 percent to about 4 percent by weight of a preservative and wherein the weight ratio of poly(N-vinyl lactam) to chitosan derivative is in the range of from about 80/1 to about 2/1.

70. (EXAMPLE 2) A composition of claim 47 wherein the poly(N-vinyl lactam) is polyvinylpyrrolidone and the weight ratio of polyvinylpyrrolidone to chitosan derivative is in the range of about 17.5/1.

71. A composition comprising a hydrogel formed by the mixture of a water soluble multifunctional amine-containing polymer and a chitosan derivative or mixtures thereof.

72. The composition of claim 71 wherein the multifunctional amine-containing polymer is selected from the group consisting of polyethyleneimine, amine terminated polyethylene oxide polymers, amine terminated polyethylene/polypropylene oxide polymers, polymers and copolymers of dimethyl amino ethyl methacrylate, and vinyl pyrrolidone.

73. The composition of claim 71 wherein the chitosan derivative is a biocompatible salt.

74. The composition of claim 73 wherein the chitosan salt is selected from the group consisting of chitosan reacted with a reactant selected from the group consisting of pyrrolidone carboxylic acid, glutamic acid and an acetate.

75. The composition of claim 72 wherein the chitosan is selected from the group consisting of N,O-carboxymethyl chitosan, and N,O-carboxybutyl chitosan.

76. The composition of claim 72 further including biologically active and pharmaceutically acceptable substances having curative or therapeutic value.

77. The composition of claim 76 wherein the active materials are selected from the group consisting of hypnotics, sedatives, tranquilizers, anti-convulsants, muscle relaxants, analgesics, antipyretic agents, anti-inflammatory agents, local anesthetics, antispasmodics, anti-ulcer agents, anti-virals, anti-bacterials, anti-fungals, sympathomimetic agents, cardiovascular agents and antitumor agents.

78. The composition of claim 77 wherein said active materials are selected from the group consisting of nitroglycerine, scopolamine, pilocarpine, ergotamine tartrate, phenylpropanolamine, theophylline, antimicrobials tetracycline, neomycin, oxytetracycline, triclosan, sodium cefazolin, silver sulfadiazine, methylsalicylate, salicylic acid, nicotinates, methyl nicotinate, chlorhexidine gluconate, menthol, capsicum and benzocaine.

79. The composition of claim 71 further including an electrolyte whereby said gel is rendered electrically conductive.

80. The composition of claim 79 wherein said electrolyte is selected from the group consisting of sodium chloride, potassium chloride and magnesium acetate.

81. The composition of claim 72 further including a skin-hydrating agent.

82. The composition of claim 81 wherein said skin-hydrating agent is selected from the group consisting of water, sodium pyrrolidone carboxylate, lactic acid, hyaluronic acid and hydrolyzed collagen.

83. The composition of claim 72 further including enhancing agents selected from the group consisting of wetting agents, moisturizers, plasticizers, surfactants and dispersing agents.

84. The composition of claim 83 where said agents are selected from the group consisting of glycerin, propylene glycol and polyethylene glycol.

85. The composition of claim 72 further including an electrolytic salt as an anti-osmotic agent.

86. The composition of claim 85 wherein said agent is selected from the group consisting of an alkali metal chloride and sodium bicarbonate.

87. The composition of claim 72 further including additives selected from the group consisting of polymer lattices, fillers, surfactants, pigments, dyes and fragrances.

88. The composition of claim 72 wherein the weight ratio of water soluble multifunctional amine-containing polymer and a chitosan derivative is in the range of 50/1 to 1/50.

89. (EXAMPLE 3) A composition of claim 72 wherein the water soluble multifunctional amine-containing polymer is polyethyleneimine and the chitosan derivative is carboxymethyl chitosan and wherein the weight ratio of said amine to said chitosan derivative is about 1.2 to 1.

90. (EXAMPLE 4) A composition of claim 72 wherein the water soluble multifunctional amine-containing polymer is polyethyleneimine and the chitosan derivative is carboxymethyl chitosan and wherein the weight ratio of said amine to said chitosan derivative is about 0.3 to 1 and further including about 13 percent by weight glycerin.

91. (EXAMPLE 5) A composition of claim 72 wherein the water soluble multifunctional amine-containing polymer is polyethyleneimine and the chitosan derivative is carboxymethyl chitosan and wherein the weight ratio of said amine to said chitosan derivative is about 0.15 to 1 and further including about 47 percent by weight glycerin.

92. The method of treating a dental dry socket which comprises applying to said socket a layer of the composition of claim 12.

93. The method of treating a dental dry socket which comprises applying to said socket a layer of the composition of claim 32.

94. The method of treating a dental dry socket which comprises applying to said socket a layer of the composition of claim 44.

95. The method of treating a dental dry socket which comprises applying to said socket a layer of the composition of claim 45.

96. The method of treating a dental dry socket which comprises applying to said socket a layer of the composition of claim 46.

97. The method of treating a dental dry socket which comprises applying to said socket a layer of the composition of claim 58.

98. The method of treating a dental dry socket which comprises applying to said socket a layer of the composition of claim 78.

99. A dental anesthetic application comprising a composition of claim 24 further including an anesthetic selected from the group comprising lidocaine, benzocaine and Eugenol in an amount of from about 1 percent to about 30 percent by weight, moisturizers and plasticizers in the range of from about 0 percent to 50 percent by weight and preservatives in the range of from about 0 percent to about 4 percent by weight and wherein the weight ratio of non-acidic poly(N-vinyl lactam) to multifunctional amine-containing polymer is in the range of from about 80/1 to about 2/1.

100. A dental anesthetic application comprising a composition of claim 24 further including an anesthetic selected from the group comprising lidocaine, benzocaine and Eugenol in an amount of from about 2 percent to about 20 percent by weight, moisturizers and plasticizers in the range of from about 5 percent to 25 percent by weight and preservatives in the range of from about 0.01 percent to about 2 percent by weight and wherein the weight ratio of non-acidic poly(N-vinyl lactam) to multifunctional amine-containing polymer is in the range of from about 30/1 to about 5/1.

101. A dental anesthetic application comprising a composition of claim 70.

102. A dental anesthetic of claim 101 wherein the local anesthetic is selected from the group consisting of lidocaine, benzocaine and Eugenol.

103. A dental anesthetic application comprising a composition of claim 78.

104. A cosmetic face mask comprising a gel of claim 1.

105. A cosmetic face mask comprising a gel of claim 16.

106. A cosmetic face mask comprising a gel of claim 17.

107. A kit for a cosmetic gel comprising separate portions of a non-acidic poly(N-vinyl lactam) having a K value of at least 30 and a multifunctional amine-containing polymer selected from the group consisting of a multifunctional amine-containing polymer and a chitosan derivative and a separate portion containing cosmetic agents selected from the group consisting of hydrating agents, fragrances and skin nutrients with instructions as to the order of addition to and the amount of water in which to form a hydrogel, application and removal directions.

108. A hydrogel in sheet or roll form comprising a composition of claim 1 further including a releasable backing sheet.

109. The hydrogel of claim 108 wherein the releasable backing layer provides protection of the hydrogel from gases, liquid, air and the selection of area to be treated.
